(19) United States
(12) Patent Application Publication
(10) Pub. No.: US 2004/0247648 A1
(43) Pub. Date: Dec. 9, 2004

(54) Fast dissolving orally consumable films containing a modified starch for improved heat and moisture resistance

(76) Inventors: David John Fadden, Morristown, NJ (US); Neema Kulkarni, Randolph, NJ (US); Albert Sorg, Columbia, NJ (US)

Correspondence Address: PFIZER, INC., 201 TABOR ROAD, MORRIS PLAINS, NJ, 07950, US

(21) Appl. No.: 10/838,045

(22) Filed: May 3, 2004

Related U.S. Application Data
(60) Provisional application No. 60/467,339, filed on May 2, 2003.

(51) Int. Cl. A61K 9/68; A61K 9/66

(52) U.S. Cl. 424/440

(57) ABSTRACT

A consumable film adapted to adhere to and dissolve in the oral cavity of a warm-blooded animal including humans, comprising a modified starch, a pharmaceutically active agent and, optionally, at least one water soluble polymer.

### PRIORITY INFORMATION

This application claims priority to U.S. provisional application No. 60/467,339, filed May 2, 2003.

### FIELD OF THE INVENTION

The present invention is related generally to fast dissolving orally consumable films for delivering one or more pharmaceutically active agents, more particularly to fast dissolving orally consumable films containing a modified starch for improving the heat and moisture resistance of the film.

### BACKGROUND OF RELATED TECHNOLOGY

Personal care products can be formulated in a variety of dosage forms, including tablets, capsules, lozenges or strips of edible thin film compositions. Edible thin film compositions applied to the oral cavity can be designed to deliver therapeutic agents to the oral mucosa. One such example is LISTERINE POCKETPAKS™ brand oral care strip products made by Pfizer Inc of New York are successful examples of an edible film compositions effective in delivering therapeutic agents particularly antimicrobial agents in the form of a combination of essential oils.

Rapidly dissolving orally consumable films of the type described above can become viscous and sticky over time when exposed to the minimal amount of heat or moisture. Such ordinary exposure to heat or moisture can adversely affect the physical stability and composition of the film resulting in undesirable texture and appearance as well as diminished shelf life and product performance. There still remains a need in the art to develop consumable thin films, having improved product stability and resistance to heat and moisture.

### SUMMARY

An embodiment of the present invention is directed to a consumable film, which is particularly well adapted to rapidly dissolve in the mouth of a warm-blooded animal including humans. In one particular aspect of the present invention, there is provided a consumable film adapted to adhere to and dissolve in the mouth of a warm-blooded animal including humans, comprising a modified starch, a pharmaceutically active agent and, optionally, at least one water soluble polymer.

The present invention is also directed to a method of preparing a supple, non-self-adhering film especially suitable for oral delivery of pharmaceutically active agents. The method comprises preparing an aqueous phase; preparing a film-forming mixture including a modified starch and optionally, at least one water soluble polymer; combining the aqueous phase and the film forming mixture to form a hydrated polymer gel; casting the hydrated polymer gel on a substrate to form a cast gel; and drying the cast gel to form the consumable film, wherein the at least one pharmaceutically active agent is added to the aqueous phase, the hydrated polymer gel or both.

### DETAILED DESCRIPTION

An embodiment of the present invention is directed to a physiologically acceptable film that is particularly well-adapted to dissolve in the oral cavity of a warm-blooded animal including humans afflicted with a disease, symptom or condition, and adhere to the mucosa of the oral cavity. Such films are particularly suited to deliver a pharmaceutically active agent useful for treating the afflicted warm-blooded animal.

In one aspect of the present invention, there is provided a consumable film adapted to adhere to and dissolve in the oral cavity of a warm-blooded animal including humans, comprising a modified starch, a pharmaceutically active agent and, optionally, at least one water soluble polymer.

The consumable film may include one or more of the following ingredients, including, but not limited to, water, antimicrobial agents, additional film forming agents or water soluble polymers, plasticizing agents, flavorings, sulfur precipitating agents, saliva stimulating agents, cooling agents, surfactants, stabilizing agents, emulsifying agents, thickening agents, binding agents, coloring agents, triglycerides, polyethylene oxides, propylene glycols, sweeteners, fragrances, preservatives and the like, as described in co-pending application U.S. patent application Ser. No. 09/395,104, by Leung et al., filed Sep. 14, 1999, which is incorporated herein by reference in its entirety.

The term “consumable” as used herein is intended to encompass substances including edible compounds, which upon administration to a consumer, is adequately tolerated without causing undue negative effects. Consumable films are shaped and sized for administration to the oral cavity of a warm-blooded animal including humans. The films are particularly well adapted to rapidly dissolve in the mouth of the warm-blooded animal. The dissolved film adheres to the surface of the mouth, typically the roof of the mouth or the tongue, and can provide a rapid delivery system for pharmaceutically active agents.

Unless specified otherwise, the term “% by weight” as used is based on the total weight of the final product (i.e., the film) as opposed to the formulation used to produce the final product, and thus denotes the percent of the total dry weight contributed by the subject ingredient. This theoretical value can differ from the experimental value, because in practice, the film typically retains some of the water and/or other substances such as alcohols (e.g., ethanol) that may be used in preparing the final product.

In one embodiment, the consumable film includes a modified starch. It has been discovered that modified starches significantly improve the overall stability and resistance of the film to adverse factors including heat and moisture for better product performance and improved storage life. Modified starches also enable the dissolution of more solids (up to twice the amount attainable with unmodified starch) in the consumable film. Modified starches when formed into a paste in combination with water are less viscous than their unmodified counterparts, and as a consequence they can “carry” more ungelatinized starch at practical viscosities. Modified starches improve paste stability and frequently possess superior physical properties such as increased solubility, better film-forming characteristics, increased whiteness, improved gel strength, more stable viscosity, increased adhesivity, improved resistance to shear and increased resistance to freeze-thaw degradation.

The modified starches used in accordance with the present invention can be prepared by mechanically, chemically or thermally modifying unmodified starches. For example, modified starches may be prepared by chemically treating starches to produce, for example, acid treatment starches, enzyme treatment starches, oxidized starches, cross-bonding starches, and other starch derivatives. Starches suitable for modification to produce modified starches may be obtained from natural products such as corn, potatoes, tapioca as well as genetically modified forms of the same such as high amylose and waxy corn as well as sorghum varieties.

More specifically, modified starches include modified corn starches, modified tapioca starches, acid and enzyme hydrolyzed corn and/or potato starches, hypochlorite-oxidized starches, acid-thinned starches, ethylated starches, cross-bonded starches, hydroxypropylated tapioca starches, hydroxypropylated corn starches, pregelatinized modified starches, and the like. Preferred modified starches are selected from pregelatinized modified corn starches and pregelatinized modified tapioca starches.

Representative examples of commercially available modified starches useful in the present invention include PURE-COTE™ modified starches such as PURE-COTE™ B793 (a pregelatinized modified corn starch) and PURE-COTE™ B795 (a pregelatinized modified corn starch), for example, available from Grain Processing Corporation, 1600 Oregon Street, Muscatine, Iowa 52761-1494 USA. The PURE-COTE™ B793 modified starch is cold water-soluble, exhibits low viscosity in solution, and dries to a clear, flexible film. The PURE-COTE™ B793 modified starch disperses and hydrates readily in cold, warm or hot water while producing minimal foam, and the finished flexible coating or film is water soluble, strong and clear and possesses excellent sheen.

In one embodiment of the present invention, the modified starch is present in amounts ranging from about 1% to 90% by weight, in another embodiment 10% to 90% by weight, and in yet another embodiment from about 35% to 80% by weight of the film.

Modified starch may be included in the film alone or optionally in combination with an additional water soluble film forming polymer such as those selected from, for example, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, pectin, dextrin, chitin, chitosan, levan, elsinan, collagen, gelatin, zein, gluten, soy protein isolate, whey protein isolate, casein and combinations thereof. A preferred water soluble polymer is pullulan. The amount of the water soluble polymer will typically be up to about 99% by weight, preferably up to about 80% by weight, more preferably up to about 50% by weight, and most preferably up to about 40% by weight of the film.

In one embodiment, the consumable film of the present invention may comprise a modified starch in combination with a water soluble film forming polymer, such as pullulan, having good film-forming properties, and may further comprise other additives such as water, antimicrobial agents, additional film forming agents or water soluble polymers, plasticizing agents, additional flavorings, sulfur precipitating agents, saliva stimulating agents, cooling agents, surfactants, stabilizing agents, emulsifying agents, thickening agents, binding agents, coloring agents, sweeteners, fragrances and the like.

The term “pharmaceutically active agents” as used herein is intended to encompass agents other than food additives, which are administered to a warm-blooded animal, including humans to treat or prevent a disease, condition or symptom that adversely affects the warm-blooded animal. These agents are not particularly limited, however, they should be physiologically acceptable and compatible with the film. Suitable pharmaceutically active agents include, but are not limited to,

(a) antimicrobial agents such as triclosan, cetylpyridium chloride, domiphen bromide, quaternary ammonium salts, zinc compounds, sanguinarine, fluorides, alexidine, octonidine, EDTA, and the like;

(b) non-steroidal anti-inflammatory agents such as aspirin, acetaminophen, ibuprofen, ketoprofen, diflunisal, fenoprofen calcium, flurbiprofen sodium, naproxen, tolmetin sodium, indomethacin, celecoxib, valdecoxib, rofecoxib and the like;

(c) antitussives such as benzonatate, caramiphen edisylate, menthol, dextromethorphan hydrobromide, chlophedianol hydrochloride and the like;

(d) decongestants such as pseudoephedrine hydrochloride, phenylepherine hydrochloride, phenylpropanolamine, pseudoephedrine sulfate and the like;

(e) antihistamines such as brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, desloratadine, brompheniramine, dexbropheniramine, fexofenadine, cetirizine, montelukast sodium and the like;

(f) expectorants such as guaifenesin, ipecac, potassium iodide, terpin hydrate and the like;

(g) antidiarrheals such as loperamide and the like;

(h) histamine II receptor antagonists such as famotidine, ranitidine and the like;

(i) proton pump inhibitors such as omerprazole, lansoprazole and the like;

(j) general nonselective CNS depressants such as aliphatic alcohols, barbiturates and the like;

(k) general nonselective CNS stimulants such as caffeine, nicotine, strychnine, picrotoxin, pentylenetetrazol and the like;

(l) drugs that selectively modify CNS function such as phenylhydantoin, phenobarbital, primidone, carbamazepine, ethosuximide, methsuximide, phensuximide, trimethadione, diazepam, benzodiazepines, phenacemide, pheneturide, acetazolamide, sulthiame bromide, gabapentin, phenytoin and the like;

(m) antiparkinson drugs such as levodopa, amantadine and the like;

(n) narcotic-analgesics such as morphine, heroin, hydromorphone, metopon, oxymorphone, levorphanol, codeine, hydrocodone, xycodone, nalorphine, naloxone, naltrexone and the like;

(o) analgesic-antipyretics such salicylates, phenylbutazone, indomethacin, phenacetin and the like;

(p) psychopharmacological drugs such as chlorpromazine, methotrimeprazine, haloperidol, clozapine, reserpine, imipramine, tranylcypromine, phenelzine, lithium-containing drugs and the like;

(q) antianginal agents such as limaprost, nitroglycerin, nifedipine, bepridil and the like; and

(r) antimigraine drugs such as sumitriptan succinate, zolmitriptan, valproic acid eletriptan hydrobromide and the like.

The pharmaceutically active agent is employed in an effective amount, which will vary depending, in part on the active agent chosen. An “effective amount” is meant to be an amount of the active agent that is sufficient to at least reduce or relieve the condition, symptom or disease being treated, but low enough to avoid any adverse side effects. In addition to the particular active agent, the effective amount of the pharmaceutically active agent may vary with the type and/or severity of the disease, symptom or condition, the age and physical condition of the patient being treated, the duration of treatment, the nature of concurrent therapy, the specific form (i.e., salt) of the pharmaceutically active agent employed, and the particular carrier from which the pharmaceutically active agent is applied.

The amount of the pharmaceutically active agent in the formulation may be adjusted to deliver a predetermined dose of the active agent over a predetermined period of time, which may typically vary from 4 to 24 hours. For example, a preferred film of the present invention may be administered at one dose every 12 hours to deliver a pharmaceutically effective amount of the active agent such as dextromethorphan hydrochloride, for example, over a period of 12 hours to a patient in need of such administration. A typical adult dose of a pharmaceutically active agent of the present film may contain from about 1 to 130 mg, preferably from about 5 to 65 mg of the active agent (e.g., dextromethorphan hydrobromide).

Examples of doses containing specific pharmaceutically active agents that can be delivered per strip of rapidly dissolving consumable film are set forth in Table A.

TABLE A

| Pharmaceutically Active Agent | Dose | |
|---|---|---|
| Chlorpheniramine Maleate | 4-12 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Cetirizine | 5-10 | mg |
| Acrivastine | 8 | mg |
| Azatadine Maleate | 1 | mg |
| Loratadine | 5-10 | mg |
| Phenylephrine Hydrochloride | 5-10 | mg |
| Dextromethorphan Hydrobromide | 10-30 | mg |
| Sildenafil | 25-100 | mg |
| Ketoprofen | 12.5-25 | mg |
| Sumatriptan Succinate | 35-70 | mg |
| Zolmitriptan | 2.5 | mg |
| Loperamide | 2 | mg |
| Famotidine | 5-10 | mg |
| Nicotine | 1-15 | mg |
| Diphenhydramine Hydrochloride | 12.5-25 | mg |
| Pseudoephedrine Hydrochloride | 15-60 | mg |
| Atorvastatin | 5-80 | mg |
| Valdecoxib | 5-20 | mg |
| Amlodipine besylate | 2.5-10 | mg |
| Rofecoxib | 5-25 | mg |
| Setraline hydrochloride | 10-100 | mg |
| Ziprasidone | 20-80 | mg |
| Eletriptan | 10-40 | mg |
| Nitroglycerin | 0.3-0.6 | mg |

Except as otherwise noted, the amount of active agent in the film according to the present invention is designated as % by weight after the film formulation has been dried and formed into the film. Generally, the amount of the active agent used in the film may be from about 0.01% to about 80% by weight, preferably from about 2.5% to about 40% by weight, and more preferably from about 5% to about 30% by weight.

In another embodiment of the present invention, the consumable film may further include antimicrobial agents including, but not limited to, essential oils as is described in co-pending U.S. patent application Ser. No. 09/395,104, by Leung et al., filed Sep. 14, 1999, which is incorporated herein by reference in its entirety. Such essential oils may be selected from, for example, carvacrol, thymol, eucalyptol, menthol, methyl salicylate, eugenol, gerianol, verbenone and the like and combinations thereof. One of the preferred combinations of essential oils is utilized in LISTERINE® brand mouthwash and oral care strips, which are, perhaps, the most well known examples of antiseptic oral compositions that has proven effective in killing microorganisms in the oral cavity that are responsible for plaque, gingivitis and bad breath. LISTERINE® brand mouthwash and oral care strips achieve their antimicrobial effect through a combination of essential oils. These essential oils include precisely balanced amounts of thymol, methyl salicylate, menthol and eucalyptol (hereinafter “the preferred essential oils”) effective in killing the undesirable microorganisms.

The amounts of the preferred essential oils used in the film compositions can vary as long as they are in amounts sufficient to provide antimicrobial efficacy. Generally, the amount of essential oils is up to about 30% and preferably from about 0.05% to about 18% by weight of the film. In one preferred embodiment, the amount of thymol, methyl salicylate and eucalyptol is each from about 0.01% to about 4% by weight, preferably from about 0.05% to about 3.0% by weight and more preferably from about 0.07% to about 2.0% by weight of the film. Menthol may be present in an amount of from about 0.01% to about 15% by weight of the composition, preferably from about 2.0% to about 9.0% by weight and more preferably from about 3% to about 9% by weight of the film. A desirable and useful amount of essential oils including the preferred essential oils can be readily determined by those skilled in the art and may exceed the preferred amounts as long as the total essential oil content does not create processing problems such as sticking. In certain embodiments, the essential oils are combined in amounts synergistically effective to kill plaque-producing germs that cause dental plaque, gingivitis and bad breath.

For embodiments incorporating essential oils, humectants are avoided due to the relatively high content of oil in the consumable, so as to avoid producing an overly moist, self-adhering film. In an embodiment, the consumable film includes a plasticizing agent other than glycerin, which is also a humectant, and with a sweetener other than sorbitol, which is a mild humectant.

Saliva stimulating agents may also be added to the consumable films of the present invention. Useful saliva stimulating agents are disclosed in U.S. Pat. No. 4,820,506, which is incorporated herein by reference in its entirety.

Suitable sweeteners include both natural and artificial sweeteners such as A) water-soluble sweeteners including monosaccharides, disaccharides, polysaccharides and the like, B) water-soluble artificial sweeteners including soluble saccharin salts and the like, C) dipeptide based sweeteners such as L-aspartic acid derived sweeteners including aspartame, neotame and the like, D) derivatives of naturally occurring water-soluble sweeteners including chlorinated derivatives of sucrose, sucralose and the like, E) protein based sweeteners including thaumatoccous danielli (Thaumatin I and II) and the like, and combinations thereof.

In general, an effective amount of auxiliary sweetener is utilized to provide the level of sweetness desired for a particular composition, and this amount will vary with the particular sweetener selected. The effective amount will normally be from about 0.01% to about 10% by weight of the consumable film when using an easily extractable sweetener. The water-soluble sweeteners are usually used in amounts of from about 0.01% to about 10% by weight, and preferably in amounts of from about 2.0% to about 5.0% by weight of the consumable film. The other sweeteners described above, other than water-soluble sweeteners are generally used in amounts of from about 0.01% to about 10% by weight, preferably from about 2% to about 8% by weight, and more preferably from about 3% to about 6% by weight of the consumable film.

A preservative may also be added to the consumable films. The preservative is added in amounts from about 0.001% to about 5%, preferably from about 0.01% to about 1% by weight of the consumable film. Preferred preservatives include sodium benzoate, potassium sorbate and the like, and combinations thereof. Other suitable preservatives include, but are not limited to, salts of edetate, (also known as salts of ethylenediaminetetraacetic acid, or EDTA, such a disodium EDTA).

Further embodiments of the present invention are directed to methods of preparing consumable films of the present invention. Generally, a pharmaceutically active agent is dissolved in water to form an aqueous phase. The aqueous phase may further include sweeteners, dyes, preservatives, food additives and the like. A film forming mixture comprising a modified starch, and optionally, at least one water soluble polymer (e.g., pullulan), is prepared. The aqueous phase and the film forming mixture are combined and thoroughly mixed to form a hydrated polymer gel. Alternatively, the pharmaceutically active agent may be added directly to the hydrated polymer gel. Optionally, an organic phase comprising organic ingredients such as essential oils and other oils (e.g. glycerine, olive oil) flavorants, surfactants (e.g., Polysorbate 80, Atmos 300, Atsurf 596K); and the like, may be combined with the aqueous phase, the film forming mixture or the hydrated polymer gel. The resulting hydrated polymer gel is cast on a suitable substrate to form a cast gel. The cast gel is then dried to form the consumable film.

In a preferred method of preparing the consumable film, it is desirable to first form the film forming mixture by first hydrating the modified starch and the optional water soluble polymer with water. The aqueous phase is then prepared by dissolving the other water soluble ingredients such as a water soluble pharmaceutically active agent, sweeteners, dyes, food additives and the like in water. Separately, the organic ingredients such as essential oils and other oils (e.g. glycerine, olive oil) flavorants, surfactants (e.g., Polysorbate 80, Atmos 300, Atsurf 596K); and the like are mixed together. The final formulation is then produced by mixing the film forming polymer phase with the aqueous phase, then adding the organic phase. The combined mixture is formed into an emulsion or a hydrated polymer gel.

The resulting hydrated polymer gel is then cast on a suitable substrate and dried to form a film. The film is preferably air-dried and dried under warm air and cut to a desired dimension, packaged and stored. The packaged film may contain moisture in amounts of from about 0.1% to 10% by weight, and more preferably from about 4% to 7% by weight.

The film forming mixture may further include stabilizing agents such as guar gum, xanthan gum, locust bean gum, carrageenan and the like, and combinations thereof. These ingredients are mixed and then hydrated in warm water, preferably deionized water until a gel is formed which may take from about 30 to 48 hours. The water is preferably heated to a temperature of from about 20° C. to 40° C. to promote hydration. The amount of water is typically from about 40% to 80% by weight of the gel. The resulting hydrated gel is then chilled to a temperature of from about 20° C. to 30° C. for about 1 to 48 hours.

The aqueous phase may, in addition to the pharmaceutically active agent, include other additives such as coloring agents, copper gluconate and sweetener. Typically the aqueous phase contains from about 5% to 80% by weight based on the total weight of the final gel mixture.

If sodium saccharin as a selected sweetener and copper gluconate as a selected sulfur precipitating agent are used in the formulation, it is preferable to dissolve them separately in solution to avoid precipitation.

In a more preferred method, the water soluble polymer is preferably in the form of a powder which is added to the aqueous phase to form a hydrated polymer gel. The resulting hydrated polymer gel is thoroughly stirred at about room temperature for about 30 minutes to 48 hours, and then deaerated to remove at least substantially all the air bubbles. The uniform mixture is cast on a suitable substrate, and thereafter dried to form the desired film.

For consumable films containing essential oils, the method of preparing consumable films of the present invention further includes adding the essential oils to the organic phase and the mixing the organic phase with the hydrated polymer gel. In particular, the essential oils such as menthol and thymol can be mixed optionally in combination with oils to form an oil mixture. Other essentials oils such as methyl salicylate and eucalyptol, and surfactants can then be added to the oil mixture. The oil mixture is then added to the hydrated polymer gel and mixed until a uniform gel is formed. The uniform gel is then cast on a suitable substrate, and thereafter dried to form the consumable film.

In one preferred method of preparing the consumable film, the modified starch and the optional water soluble polymer may be hydrated without heating the water to reduce energy costs in the manufacturing process. Moreover, heating results in undesirable losses of volatile ingredients to evaporation. For essential oil-containing films of the present invention, the heat may also affect the germ killing activity of the composition due to the loss of essential oils. The essential oils may be mixed to minimize loss of flavor.

While not wishing to be bound by any theory, it is believed that the film forming ingredients such as the modified starch and the optional water soluble polymers can be hydrated and mixed without heating due to an ionic effect known as the Donnan equilibrium. Hydrating the modified starch and the optional water soluble polymers in the presence of electrolytes in solution effectively lowers the viscosity of the polymer gel being formed, thus increasing the efficiency of the hydrating process. The water-soluble ingredients of the formulation provide the electrolytes, which are dissolved in the hydration solution prior to addition to the modified starches and the optional water-soluble polymers. High shear mixing also accelerates hydration, which delumps the powders, providing greater surface area for water contact. In addition, local heating effects, generated in the shear regions, provide energy for hydration without substantially raising the temperature of the mass.

### EXAMPLE 1

### Quick Dissolving Consumable Film Containing 15.0 mg Dextromethorphan Hydrobromide and PURE-COTE™ B793 Base

The ingredients listed in Table 1 were combined to provide a consumable film of the present invention in accordance with the following procedure:

A) Dextromethorphan HBr was mixed and dissolved in 90% water at 75° C. to yield an aqueous phase. Amberlite IRP69 was added to the aqueous phase and stirred for about 4 to 5 hours at about 70° C. to 80° C. Pectin was added to the aqueous phase very slowly and mixed at a high mixing rate. The aqueous phase was allowed to cool to about 50° C. and q.s. with water to replace loss due to evaporation. Potassium sorbate and dye were then added to the aqueous phase and mixed thoroughly.

B) The film-forming ingredients, xanthan gum, locust bean gum, carrageenan and PURE-COTE™ B793 were mixed together in a separate container to form a film forming mixture.

C) The film forming mixture was slowly added to the aqueous phase of A), followed by overnight mixing at a low mixing rate to provide a hydrated polymer gel.

D) The flavorants, glycerine, olive oil, menthol, and surfactants were combined and mixed to dissolve in a separate container to yield an organic phase.

E) Mannitol and sucralose were mixed together in the remaining 10% water in a separate container. Succulence was then added to the resulting mixture and dissolved.

F) The mixtures of steps D) and E) were added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 1

| | | | % w/w | |
|---|---|---|---|---|
| | | % w/w* | Actual | |
| Material | mg/dose* | Dry Film | Batch | g/batch |
| Dextromethorphan HBr | 15.0000 | 19.5740 | 10.6759 | 106.7593 |
| Amberlite IRP69 | 16.0001 | 20.8790 | 11.3877 | 113.8771 |
| Pectin USP | 0.3499 | 0.4566 | 0.2490 | 2.4905 |
| Xanthan Gum | 0.0769 | 0.1003 | 0.0547 | 0.5470 |
| Locust Bean Gum | 0.0901 | 0.1175 | 0.0641 | 0.6409 |
| Carrageenan | 0.3860 | 0.5037 | 0.2747 | 2.7474 |
| PURE-COTE ™ B793 | 20.5919 | 26.8711 | 14.6559 | 146.5586 |
| Potassium sorbate | 0.0772 | 0.1008 | 0.0550 | 0.5498 |
| Purified water | — | — | 45.4586 | 454.5856 |
| Menthol | 2.5740 | 3.3589 | 1.8320 | 18.3202 |
| Peppermint Flavor | 0.2579 | 0.3366 | 0.1836 | 1.8357 |
| Cherry Flavor (Givudan) | 0.2579 | 0.3366 | 0.1836 | 1.8357 |
| Sour Cherry (IFF) | 2.2350 | 2.9165 | 1.5907 | 15.9070 |
| Warm Sensation (Mane) | 0.5518 | 0.7200 | 0.3927 | 3.9270 |
| Artificial Masking Agent | 0.4140 | 0.5402 | 0.2946 | 2.9463 |
| Flavor (Robertet) | | | | |
| Succulence (IFF) | 0.2579 | 0.3366 | 0.1836 | 1.8357 |
| FD&C Red #40 | 0.0099 | 0.0129 | 0.0070 | 0.0704 |
| Polysorbate 80 NF | 0.4505 | 0.5878 | 0.3206 | 3.2060 |
| Atmos 300 | 0.4505 | 0.5878 | 0.3206 | 3.2060 |
| Glycerine | 8.7335 | 11.3966 | 6.2158 | 62.1585 |
| Olive Oil | 3.49934 | 4.5586 | 2.4863 | 24.8634 |
| Mannitol USP | 2.5740 | 3.3589 | 1.8320 | 18.3202 |
| Sucralose | 1.8001 | 2.3490 | 1.2812 | 12.8116 |
| Total | 76.6324 | 100.0000 | 100.0000 | 1000.0000 |

*Assuming that all water is evaporated

### EXAMPLE 2

### Quick Dissolving Consumable Film Containing 15.0 mg Dextromethorphan Hydrobromide and PURE-COTE™ B793 Base

The ingredients listed in Table 2 were combined to provide a consumable film of the present invention in accordance with the procedure of Example 1, except having Atmos 300 substituted by Atsurf 596K.

TABLE 2

| | | | % w/w | |
|---|---|---|---|---|
| | | % w/w* | Actual | |
| Material | mg/dose* | Dry Film | Batch | g/batch |
| Dextromethorphan HBr | 15.0000 | 18.5409 | 10.3611 | 103.6107 |
| Amberlite IRP69 | 16.0001 | 19.7771 | 11.0519 | 110.5186 |
| Pectin USP | 0.3499 | 0.4325 | 0.2417 | 2.41 70 |
| Xanthan Gum | 0.0769 | 0.0950 | 0.0531 | 0.5309 |
| Locust Bean Gum | 0.0901 | 0.1113 | 0.0622 | 0.6220 |
| Carrageenan | 0.3860 | 0.4771 | 0.2666 | 2.6664 |
| PURE-COTE ™ B793 | 20.5919 | 25.4529 | 14.2236 | 142.2363 |
| Potassium sorbate | 0.0772 | 0.0955 | 0.0534 | 0.5335 |
| Purified water | — | — | 44.1179 | 451.1788 |
| Menthol | 2.5740 | 3.1817 | 1.7780 | 17.7799 |
| Peppermint Flavor | 0.2579 | 0.3188 | 0.1782 | 1.7816 |
| Cherry Flavor (Givudan) | 0.2579 | 0.3188 | 0.1782 | 1.7816 |
| SourCherry (IFF) | 2.2350 | 2.7626 | 1.5438 | 15.4379 |
| Warm Sensation (Mane) | 0.5518 | 0.6820 | 0.3811 | 3.8112 |
| Artificial Masking Agent | 0.4140 | 0.5117 | 0.2859 | 2.8594 |
| Flavor (Robertet) | | | | |
| Succulence (IFF) | 0.2579 | 0.3188 | 0.1782 | 1.7816 |
| FD&C Red #40 | 0.0099 | 0.0122 | 0.0068 | 0.0684 |
| Polysorbate 80 NF | 0.4505 | 0.5568 | 0.3111 | 3.1114 |
| Atmos 300 | 0.4505 | 0.5568 | 0.3111 | 3.1114 |
| Glycerine | 11.6446 | 14.3935 | 8.0434 | 80.4337 |
| Olive Oil | 4.8519 | 5.9973 | 3.3514 | 33.5140 |
| Mannitol USP | 2.5740 | 3.1817 | 1.7780 | 17.7799 |
| Sucralose | 1.8001 | 2.2250 | 1.2434 | 12.4337 |
| Total | 80.9021 | 100.0000 | 100.0000 | 1000.0000 |

*Assuming that all water is evaporated

### EXAMPLE 3

### Vanilla Mint Flavored Quick Dissolving Consumable Film Containing 10 mg Famotidine Hydrochloride and PURE-COTE™ B793 Base

The ingredients listed in Table 3 were combined to provide a consumable film of the present invention in accordance with the following procedure:

A) Potassium sorbate and dye were mixed in 80% water.

B) The film-forming ingredients, xanthan gum, locust bean gum, carrageenan and PURE-COTE™ B793 were mixed together in a separate container to form a film forming mixture.

C) The film forming mixture was slowly added to the mixture of A), followed by overnight mixing at a low mixing rate to form a hydrated polymer gel.

D) Mannitol and sucralose were mixed together with remaining 20% of water in a separate container, and then added to the hydrated polymer gel and mixed well.

E) Milled famotidine HCl was added to the hydrated polymer gel and mixed thoroughly.

F) The flavorants, glycerine, olive oil and surfactants were combined and mixed thoroughly in a separate container.

G) The resulting mixture of step F) was added to the hydrated polymer gel and mixed uniformly to yield a final polymer gel mixture. The final polymer gel mixture was poured on a mold and cast to form a film of a desired thickness at room temperature. The film was dried under warm air and cut to a desired dimension (dictated by e.g., dosage and mouthfeel).

TABLE 3

| | | | % w/w | |
|---|---|---|---|---|
| | | % w/w* | Actual | |
| Material | mg/dose* | Dry Film | Batch | g/batch |
| Famotidine HCI | 10.0000 | 15.2065 | 5.3223 | 106.4453 |
| Xanthan Gum | 0.1154 | 0.1754 | 0.0614 | 1.2278 |
| Locust Bean Gum | 0.1352 | 0.2055 | 0.0719 | 1.4386 |
| Carrageenan | 0.5792 | 0.8807 | 0.3082 | 6.1648 |
| PURE-COTE ™ B793 | 30.8879 | 46.9695 | 16.4393 | 328.7865 |
| Potassium sorbate | 0.1158 | 0.1761 | 0.0616 | 1.2326 |
| Purified water | — | — | 65.0000 | 1300.0000 |
| Vanilla Mint Flavor | 2.0000 | 3.0413 | 1.0645 | 21.2891 |
| (IFF) | | | | |
| Polysorbate 80 NE | 0.6756 | 1.0273 | 0.3596 | 7.1914 |
| Atsurf 596 K | 0.6756 | 1.0273 | 0.3596 | 7.1914 |
| Glycerine | 10.0000 | 15.2065 | 5.3223 | 106.4453 |
| Olive oil | 4.0000 | 6.0826 | 2.1289 | 42.5781 |
| FD&C Blue #1 | 0.0160 | 0.0243 | 0.0085 | 0.1703 |
| Mannitol USP | 3.8610 | 5.8712 | 2.0549 | 41.0985 |
| Sucralose | 2.7000 | 4.1057 | 1.4370 | 28.7402 |
| Total | 65.7615 | 100.0000 | 100.0000 | 2000.0000 |

*Assuming that all water is evaporated

### EXAMPLE 4

### Vanilla Mint Flavored Quick Dissolving Consumable Film Containing 10 mg Famotidine Hydrochloride and a Modified Tapioca Starch Base

The ingredients listed in Table 4 were combined to provide a consumable film of the present invention in accordance with the procedure of Example 3, except having PURE-COTE™ B793 substituted by a modified tapioca starch.

TABLE 4

| | | % w/w* | % w/w | |
|---|---|---|---|---|
| Material | mg/dose* | Dry Film | Actual Batch | G/batch |
| Famotidine HCl | 10.0000 | 9.7503 | 4.4512 | 26.6184 |
| Xanthan Gum | 0.1154 | 0.1125 | 0.0513 | 0.3070 |
| Locust Bean Gum | 0.1352 | 0.1318 | 0.0602 | 0.3597 |
| Carrageenan | 0.5792 | 0.5647 | 0.2578 | 1.5416 |
| Tapioca Starch J474 | 67.6870 | 65.9970 | 30.1291 | 180.1720 |
| Potassium sorbate | 0.1158 | 0.1129 | 0.0515 | 0.3082 |
| Purified water | — | — | 54.3478 | 324.9998 |
| Vanilla Mint | 2.0000 | 1.9501 | 0.8902 | 5.237 |
| Flavor (IFF) | | | | |
| Polysorbate 80 NF | 0.6756 | 0.6587 | 0.3007 | 1.7983 |
| Atsurf 596K | 0.6756 | 0.6587 | 0.3007 | 1.7983 |
| Glycerine | 10.0000 | 9.7503 | 4.4512 | 26.6184 |
| Olive oil | 4.0000 | 3.9001 | 1.7805 | 10.6474 |
| FD&C Blue #1 | 0.0160 | 0.0156 | 0.0071 | 0.0426 |
| Mannitol USP | 3.8610 | 3.7646 | 1.7186 | 10.2774 |
| Sucralose | 2.7000 | 2.6326 | 1.2018 | 7.1870 |
| Total | 102.5607 | 100.0000 | 100.0000 | 598.0000 |

*Assuming that all water is evaporated

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

We claim:

1. A consumable film adapted to adhere to and dissolve in the oral cavity of a warm-blooded animal including humans, comprising a modified starch and a pharmaceutically active agent.

2. The consumable film of claim 1 wherein the modified starch is selected from the group consisting of a mechanically modified starch, a chemically modified starch, a thermally modified starch and combinations thereof.

3. The consumable film of claim 1 wherein the modified starch is selected from a chemically modified starch.

4. The consumable film of claim 1 wherein the modified starch is selected from the group consisting of modified corn starches, modified tapioca starches, acid hydrolyzed corn starches, acid hydrolyzed potato starches, enzyme hydrolyzed corn starches, enzyme hydrolyzed potato starches, hypochlorite-oxidized starches, acid-thinned starches, ethylated starches, cross-bonded starches, hydroxypropylated tapioca starches, hydroxypropylated corn starches, pregelatinized modified starches and combinations thereof.

5. The consumable film of claim 1 wherein the modified starch is selected from the group consisting of a pregelatinized modified corn starch, a pregelatinized modified tapioca starch and combinations thereof.

6. The consumable film of claim 1 wherein the modified starch is a pregelatinized modified starch.

7. The consumable film of claim 1 wherein the modified starch is present in the amount of from about 1% to 90% by weight based on the total weight of the consumable film.

8. The consumable film of claim 1 wherein the pharmaceutically active agent is selected from the group consisting of benzonatate, caramiphen edisylate, menthol, dextromethorphan hydrobromide, chlophedianol hydrochloride and combinations thereof.

9. The consumable film of claim 1 wherein the pharmaceutically active agent is selected from the group consisting of pseudoephedrine hydrochloride, phenylepherine hydrochloride, phenylpropanolamine, pseudoephedrine sulfate and combinations thereof.

10. The consumable film of claim 1 wherein the pharmaceutically active agent is selected from the group consisting of brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, brompheniramine, dexbropheniramine, fexofenadine, cetirizine and combinations thereof.

11. The consumable film of claim 1 wherein the pharmaceutically active agent is selected from the group consisting of famotidine, ranitidine and combinations thereof.

12. The consumable film of claim 1 wherein the pharmaceutically active agent is selected from the group consisting of aspirin, acetaminophen, ibuprofen, ketoprofen, diflunisal, fenoprofen calcium, naproxen, tolmetin sodium, indomethacin, flurbiprofen sodium, celecoxib, valdecoxib, rofecoxib and mixtures thereof.

13. The consumable film of claim 1 further comprising at least one water soluble polymer; wherein the at least one water soluble polymer is selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and combinations thereof.

14. The consumable film of claim 1, wherein said film is in the form of a single layer.

15. A method for delivering and enhancing the retention of a pharmaceutically active agent to the oral cavity of a warm-blooded animal including humans, comprising orally administering the consumable film of claim 1 to said warm-blooded animal.
